(19) United States
(12) Patent Application Publication
(10) Pub. No.: US 2024/0288369 A1
(43) Pub. Date: Aug. 29, 2024

(54) Apparatus and Method for Optically Characterizing a Textile Sample

(71) Applicant: Uster Technologies AG, Uster (CH)

(72) Inventors: Peyman H. Dehkordi, Knoxville, TN (US); Kent A. Rinehart, Knoxville, TN (US); David Dickson McAlister, III, Knoxville, TN (US)

(73) Assignee: Uster Technologies AG, Uster (CH)

(21) Appl. No.: 18/659,638

(22) Filed: May 9, 2024

Related U.S. Application Data
(63) Continuation of application No. 17/632,429, filed on Feb. 2, 2022, now Pat. No. 12,013,340.

(51) Int. Cl. G01N 21/64; G01N 21/17; G01N 21/25; G01N 21/27; G01N 21/31; G01N 21/47; G01N 21/55; G01N 33/36

(52) U.S. Cl. CPC G01N 21/6456; G01N 21/256; G01N 21/276; G01N 21/31; G01N 21/55; G01N 33/367; G01N 2021/174; G01N 2021/178; G01N 2021/3155; G01N 2021/4766; G01N 2021/6423; G01N 2021/6471

(57) ABSTRACT

An apparatus (100) for optically characterizing a textile sample (106) comprises a presentation subsystem (102) comprising a viewing window (108). A radiation subsystem (114) comprises a radiation source (120) for directing a first, ultraviolet radiation (122) and a second, visible radiation (123) toward the sample (106), and causing the sample (106) to produce a fluorescent radiation (124) and a reflected radiation (125). A sensing subsystem (126) comprises an imager (130) for capturing the fluorescent radiation (124) and the reflected radiation (125) in an array of pixels (408). A control subsystem (132) comprises a processor (136) for controlling the presentation subsystem (102), the radiation subsystem (114), and the sensing subsystem (126), and for creating a fluorescent and reflected radiation image (400) containing both spectral information and spatial information in regard to the fluorescent radiation (124) and the reflected radiation (125).

### FIELD

This invention relates to the field of textile characteristics measurement. More particularly, this invention relates to an apparatus and a method for optically characterizing a textile sample. The invention can be used in online and offline applications.

### INTRODUCTION

Textiles are woven or knitted fabrics made from yarn, but they also include fibers (natural, manmade, and blend), yarn, or any other product made from these combinations. The visual perception of the final fabric is very important to the end user. Visual perception includes, but is not limited to, pattern, color, and brightness. Brightness is commonly defined as an attribute of visual perception in which a source appears to be radiating or reflecting light. For textile, the brightness is a function of reflected light and fluorescence emission. Fluorescent pigments are one method of adding brightness to the fabric. A fluorescent material is defined as a material that emits optical radiation after having absorbed light or other electromagnetic radiation, typically at wavelengths that are longer than the wavelengths of the electromagnetic radiation absorbed. Therefore, fluorescent material not only reflects the incident light but also emits an additional light at longer wavelengths. Especially if the absorbed electromagnetic radiation is in the ultraviolet (UV) range and the emitted light is visible, the fluorescent material appears to be brighter.

There are at least three major categories of fluorescent pigments: inorganic fluors, optical whiteners, and daylight fluorescents. Their major differences are in their chemical composition and optical characteristics, and thus their application. For example, inorganic fluors are usually activated by optical radiations in the range of ultraviolet (UV) to visible (300-420 nm) light, whereas optical whiteners are activated in the range of near UV (340-400 nm), and daylight fluorescents are activated and emit in the visible range (400-700 nm).

Fluorescent properties can be given to textiles in a variety of ways, such as, but not limited to (a) coating fabrics with fluorescent material in a resin mix, (b) introducing fluorescent material to the fibers at the spinning stage, (c) coating the fibers, and (d) textile finishing or domestic laundering with fluorescent material.

The quality and application of the fluorescent pigments are important parameters to maintain, monitor, and control for best cosmetic performance in the textile process. While these methods have been around for many years, the formulation and quality control of the fluorescent material application have been challenging, primarily due to lack of proper measurement instrumentation and lack of understanding of the fluorescent material.

US-2015/0131090 A1 discloses a multi-angle spectral imaging measurement method and apparatus. A lighting device that emits illumination light from two or more angular directions onto a sample surface to be measured, an imaging optical lens, and a monochrome two-dimensional image sensor are provided. This configuration provides a method and an apparatus that take a two-dimensional image of the sample surface to be measured at each measurement wavelength and accurately measures multi-angle and spectral information on each of all pixels in the two-dimensional image in a short time. In one embodiment, a fluorescent object is illuminated with monochromatic light and a spectral light receiver is used so that fluorescent color can be measured.

US-2016/0258881 A1 discloses a method of marking an industrial process material including selectively incorporating a luminescent marker onto and/or into the industrial process material in a trace amount insufficient to be optically detectable in the presence of ambient light but sufficient to be non-destructively optically detectable in and/or on the industrial process material in situ in the field or on-site. The material is illuminated with light of a certain wavelength. When the luminescent marker is present in the material, it emits luminescent light of a characteristic wavelength. Light emitted by the material is detected by a spectrometer that can include a CCD chip as the light-sensitive element.

### SUMMARY

It is an object of the present invention to characterize even more completely a textile sample with regard to its optical properties.

This and other objects are achieved by the apparatus and the method defined in the independent claims. The dependent claims define preferred embodiments.

According to the invention, the textile sample is illuminated with both ultraviolet (UV) and visible electromagnetic radiation. The fluorescent radiation emitted due to the UV radiation and the reflected visible radiation are both detected and combined to create a “brightness” measurement. Moreover, both the fluorescent and the reflected radiation are detected by means of an imager comprising an array of pixels. Thus, the fluorescent radiation, the reflected radiation and their combination bear embedded spatial information. In particular, the resulting brightness image turned out to be very useful for optically characterizing the textile sample.

In this document, the term “brightness” designates an intensity of both fluorescent and reflected radiation. As generally accepted, the ultraviolet range of the electromagnetic spectrum contains wavelengths between 10 nm and 400 nm, whereas the visible range contains wavelengths between 400 nm and 700 nm.

The apparatus for optically characterizing a textile sample comprises a presentation subsystem having a viewing window. A radiation subsystem has a radiation source for directing a desired first radiation in the ultraviolet range of the electromagnetic spectrum and a desired second radiation in the visible range of the electromagnetic spectrum through the viewing window toward the sample, and thereby causing the sample to produce both emission fluorescent radiation and a reflected radiation. A sensing subsystem comprises an imager for capturing the fluorescent radiation and the reflected radiation in an array of pixels, where each pixel records an intensity of both the fluorescent radiation and the reflected radiation at the pixel location. A control subsystem comprises a processor for controlling the presentation subsystem, the radiation subsystem, and the sensing subsystem, and creates a fluorescent and reflected radiation image containing both spectral information and spatial information in regard to the fluorescent radiation and the reflected radiation of the sample.

In some embodiments, the presentation subsystem further comprises a sample press for pressing the sample against the viewing window.

In some embodiments, the presentation subsystem further comprises calibration tiles for producing fluorescent radiation and reflected radiation with known characteristics in response to radiation having known characteristics.

In some embodiments, the radiation subsystem further comprises optics for shaping and/or filtering the first radiation and the second radiation from the radiation source to produce the first radiation and/or the second radiation.

In some embodiments, the radiation subsystem further comprises a detector for detecting characteristics of the first radiation and/or the second radiation.

In some embodiments, the radiation source is configured to produce the first radiation and the second radiation at a desired intensity distribution.

In some embodiments, the radiation source is configured to produce the first radiation and the second radiation in discrete radiation ranges.

In some embodiments, the radiation source is configured to produce the first radiation and the second radiation with an intensity distribution that varies in time.

In some embodiments, the radiation source is configured to produce the first radiation and the second radiation sequentially.

In some embodiments, the radiation source is configured to produce the first radiation and the second radiation simultaneously.

In some embodiments, the radiation source is configured to produce the first radiation and the second radiation in a temporally offset way, such that they partially overlap in time.

In some embodiments, the radiation source is configured to produce the first radiation and/or the second radiation periodically in time.

In some embodiments, the array of pixels is a two-dimensional array of pixels.

In some embodiments, the sensing subsystem further comprises a variable filter for selectively prohibiting portions of the fluorescent radiation and/or the reflected radiation from achieving the imager.

In some embodiments, the control subsystem further comprises a machine interface for receiving commands from and sending information to another instrument.

In some embodiments, the control subsystem further comprises a human interface for receiving commands from and sending information to a user.

In some embodiments, the control subsystem is configured to classify patterns in the fluorescent and/or reflected radiation image, including a percentage of the fluorescent and/or reflected radiation image individually represented by each one of the patterns.

In some embodiments, the control subsystem is configured to classify patterns in the fluorescent and/or reflected radiation image, including an orientation of each one of the patterns, where the orientation is at least one of horizontal, vertical, and non-ordinal.

According to another aspect of the invention, there is described a method for optically characterizing a textile sample. The method comprises the steps of: presenting the sample against a viewing window with a presentation subsystem; directing a desired first radiation in the ultraviolet range of the electromagnetic spectrum and a desired second radiation in the visible range of the electromagnetic spectrum from a radiation source through the viewing window toward the sample, thereby causing the sample to produce a fluorescent radiation and a reflected radiation; capturing the fluorescent radiation and the reflected radiation with a sensing subsystem including an imager in an array of pixels, where each pixel records an intensity of both the fluorescent radiation and the reflected radiation at the pixel location; and with a processor, controlling the presentation subsystem, the radiation subsystem, and the sensing subsystem, and creating a fluorescent and reflected radiation image containing both spectral information and spatial information in regard to the fluorescent radiation and the reflected radiation of the sample.

Some embodiments further comprise pressing the sample against the viewing window with a press.

Some embodiments further comprise shaping and/or filtering first radiation and the second radiation from the radiation source with optics before the first radiation and/or the second radiation achieves the sample.

Some embodiments further comprise detecting characteristics of the first radiation and/or the second radiation with a detector.

Some embodiments further comprise producing the first radiation and the second radiation at a desired intensity distribution.

Some embodiments further comprise producing the first radiation and the second radiation in discrete radiation ranges.

Some embodiments further comprise producing the first radiation and the second radiation with an intensity distribution that varies in time.

Some embodiments further comprise producing the first radiation and the second radiation sequentially.

Some embodiments further comprise producing the first radiation and the second radiation simultaneously.

Some embodiments further comprise producing the first radiation and the second radiation in a temporally offset way, such that they partially overlap in time.

Some embodiments further comprise producing the first radiation and/or the second radiation periodically in time.

Some embodiments further comprise selectively prohibiting the fluorescent radiation and the reflected radiation, respectively, from achieving the imager.

Some embodiments further comprise classifying patterns in the fluorescent and/or reflected radiation image, including a percentage of the fluorescent and/or reflected radiation image individually represented by each one of the patterns.

Some embodiments further comprise classifying patterns in the fluorescent and/or reflected radiation image, including an orientation of each one of the patterns, where the orientation is at least one of horizontal, vertical, and non-ordinal.

### DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a functional block diagram of a measurement device, according to an embodiment of the present invention.

FIGS. 2A-2E are graphs of radiation source wavelength versus output level for a selection of radiation profiles, according to various embodiments of the present invention.

FIG. 3A is a fluorescence graph of radiation level versus fluorescence level for three different materials, according to an embodiment of the present invention.

FIG. 3B is a fluorescence graph of radiation level versus fluorescence level for three different radiation wavelengths, according to an embodiment of the present invention.

FIG. 3C is a fluorescence graph of radiation level versus fluorescence level for three different fluorescence wavelengths, according to an embodiment of the present invention.

FIG. 4 is an example of a fluorescence pattern, according to an embodiment of the present invention.

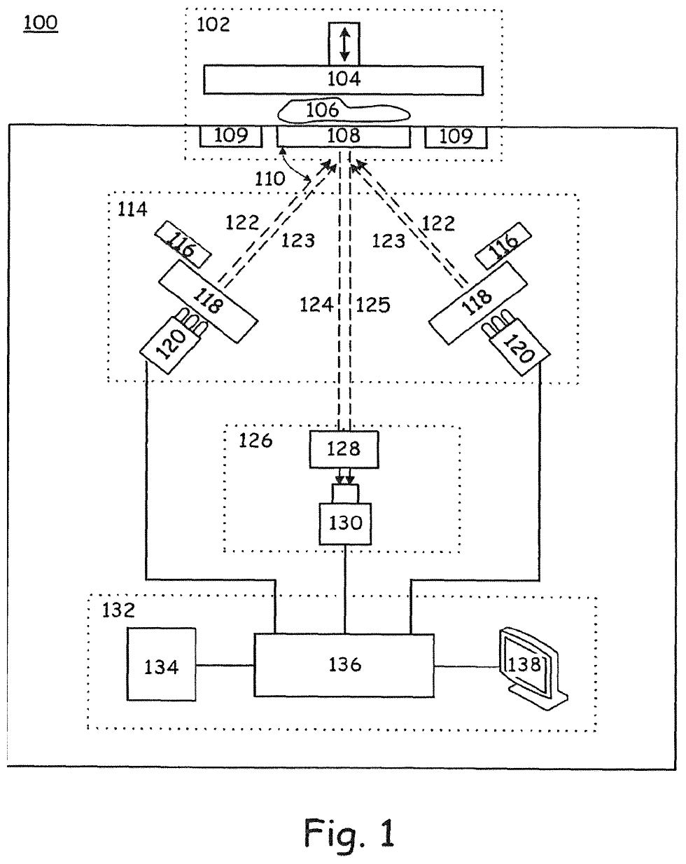

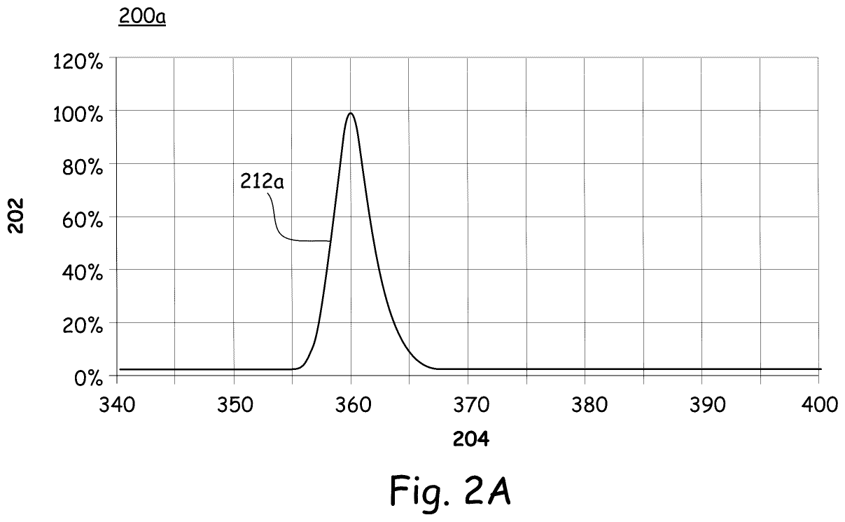

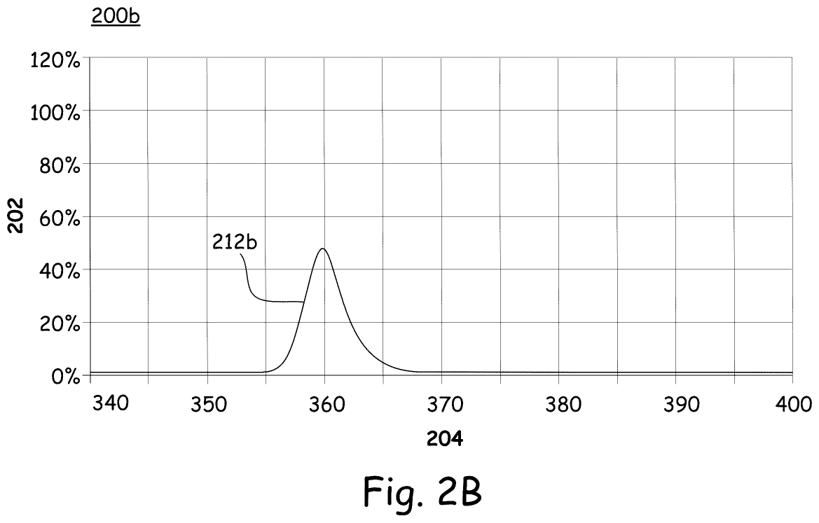

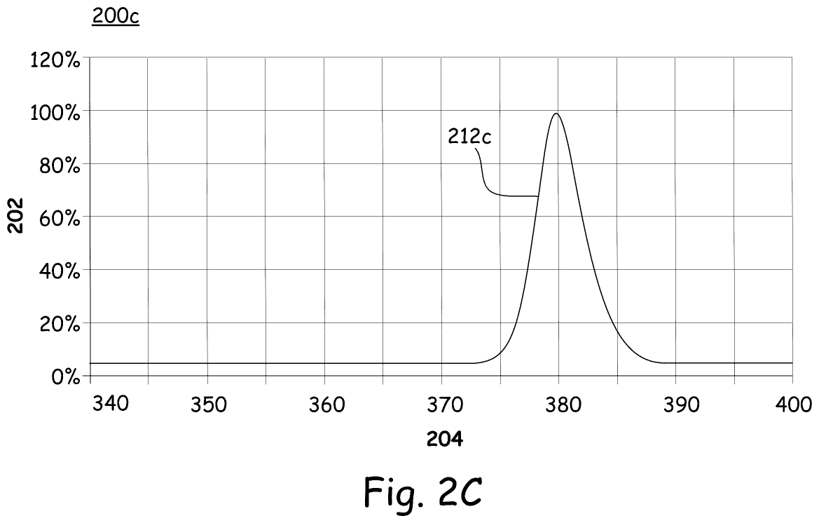

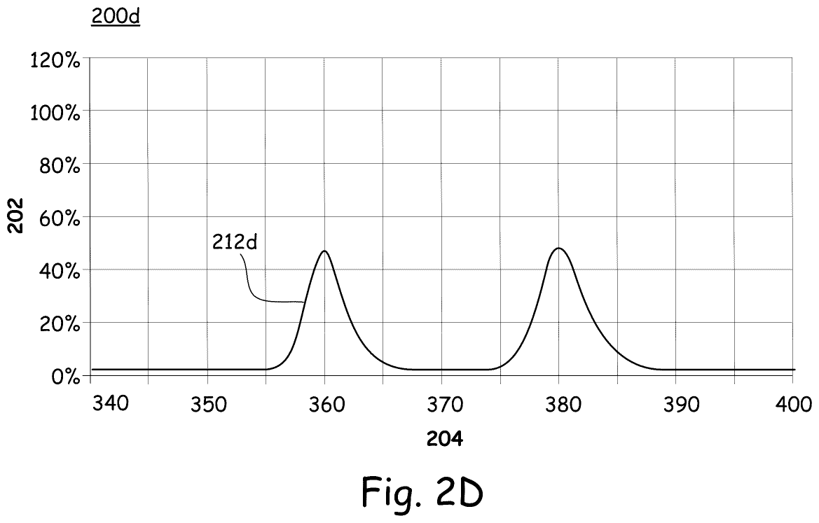

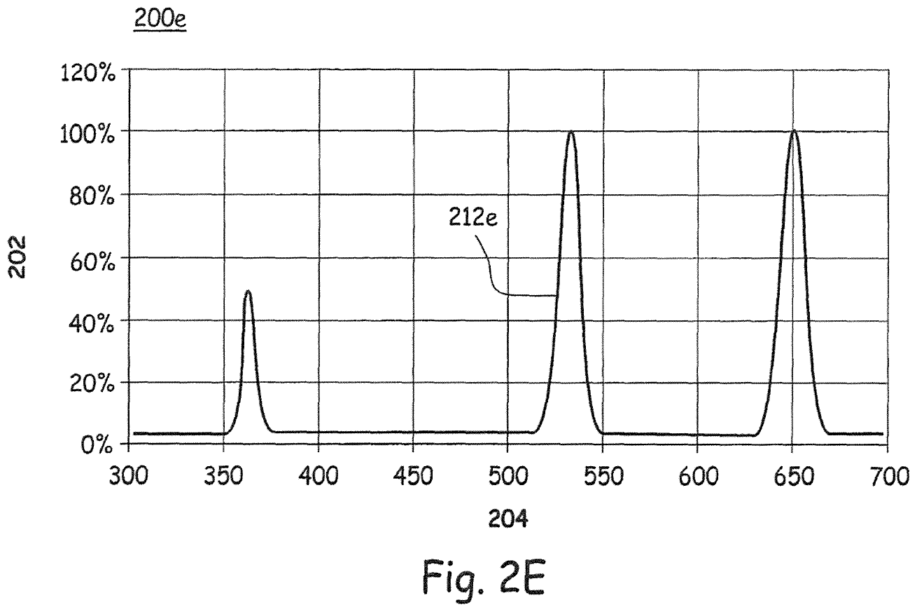

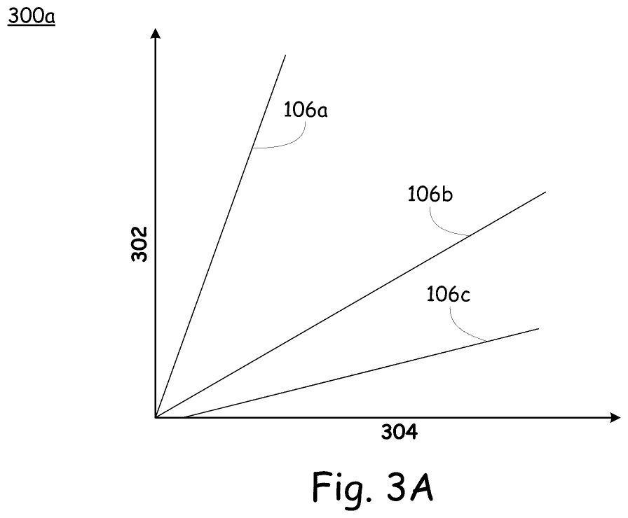

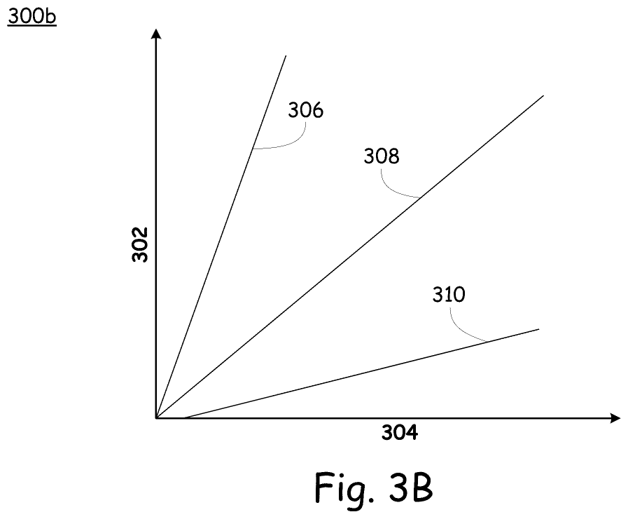

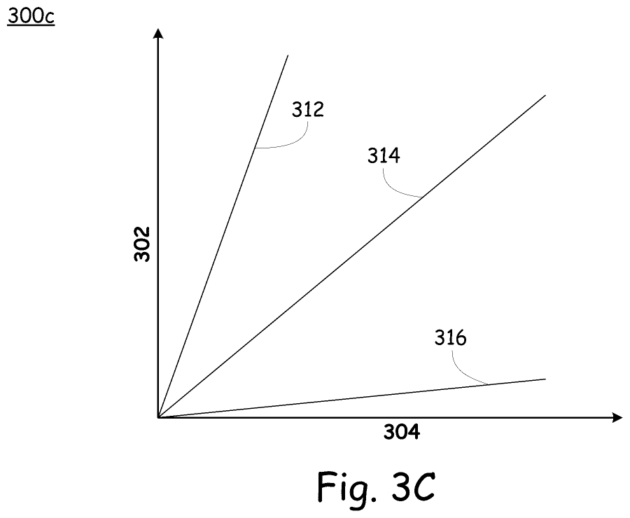

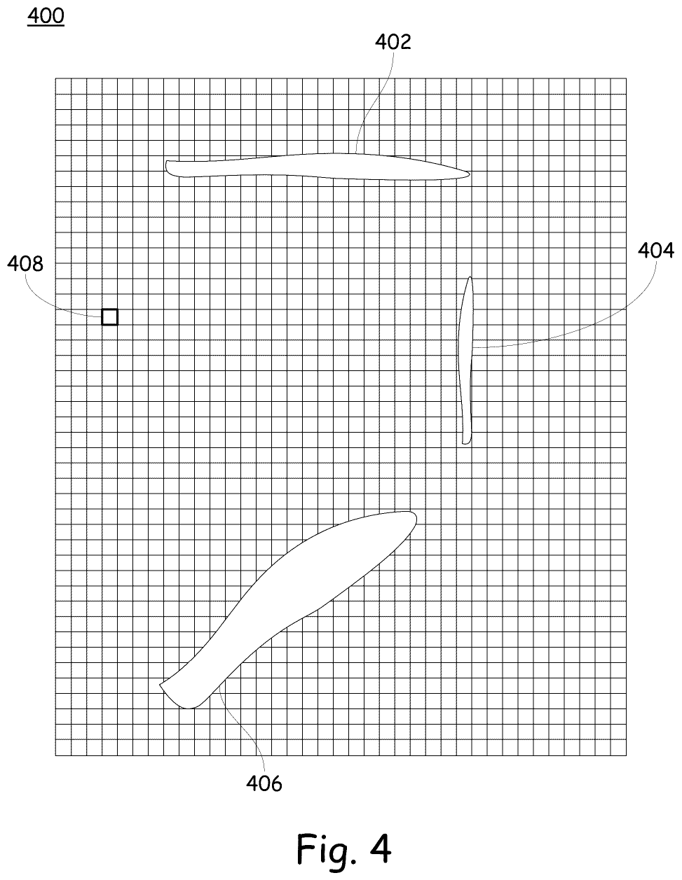

1. An apparatus (100) for optically characterizing a textile sample (106), the apparatus (100) comprising:
a presentation subsystem (102) comprising a viewing window (108),
a radiation subsystem (114) comprising a radiation source (120) for directing a desired first radiation (122) in the ultraviolet range of the electromagnetic spectrum and a desired second radiation (123) in the visible range of the electromagnetic spectrum through the viewing window (108) toward the sample (106), and causing the sample (106) to produce a fluorescent radiation (124) and a reflected radiation (125),
a sensing subsystem (126) comprising an imager (130) for capturing the fluorescent radiation (124) and the reflected radiation (125) in an array of pixels (408), where each pixel (408) records an intensity of both the fluorescent radiation (124) and the reflected radiation (125) at the pixel location, and
a control subsystem (132) comprising a processor (136) for controlling the presentation subsystem (102), the radiation subsystem (114), and the sensing subsystem (126), for creating a fluorescent and reflected radiation image (400) containing both spectral information and spatial information in regard to the fluorescent radiation (124) and the reflected radiation (125) of the sample (106), and for processing the fluorescent and reflected radiation image (400) into a brightness image
$$BI ( {λ}_{j} ) = a ( {λ}_{j} ) * FI [ \begin{matrix}{i}_{11} & … & {i}_{1 n} \\ ⋮ & {i}_{xy} & ⋮ \\ {i}_{m 1} & … & {i}_{mn}\end{matrix} ] + b ( {λ}_{j} ) * LI [ \begin{matrix}{i}_{11} & … & {i}_{1 n} \\ ⋮ & {i}_{xy} & ⋮ \\ {i}_{m 1} & … & {i}_{mn}\end{matrix} ] ,$$
where:
a(λⱼ) is weighting factor for the fluorescence image of wavelengths, where λₘᵢₙ<Δⱼ<λₘₐₓ, j=1, 2, . . . , k,
b(Δⱼ) is weighting factor for the reflected radiation image of wavelengths, where λₘᵢₙ<Δⱼ<λₘₐₓ, j=1, 2, . . . , k,
BI(λⱼ) is the brightness image of the sample 106 for one or more ranges of wavelengths, where λₘᵢₙ<Δⱼ<λₘₐₓ, j=1, 2, . . . , k,
FI(λⱼ) is the fluorescence image of the sample 106 for one or more ranges of wavelengths, where λₘᵢₙ<Δⱼ<λₘₐₓ, j=1, 2, . . . , k,
LI(Δⱼ) is the reflected radiation image of the sample 106 for one or more ranges of wavelengths, where λₘᵢₙ<Δⱼ<λₘₐₓ, j=1, 2, . . . , k,
n is the number of spatial points for the image in the horizontal direction,
m is the number of spatial points for the image in the vertical direction, and
i_{xy} is the energy level of the sample at coordinate location of x and y at wavelength Δⱼ.

2. The apparatus of claim 1, wherein the presentation subsystem (102) further comprises a sample press (104) for pressing the sample (106) against the viewing window (108).

3. The apparatus of claim 1, wherein the presentation subsystem (102) further comprises calibration tiles (109) for producing fluorescent radiation (124) and reflected radiation (125) with known characteristics in response to radiation (122) having known characteristics.

4. The apparatus of claim 1, wherein the radiation subsystem (114) further comprises optics (118) for at least one of shaping or filtering the first radiation (122) and the second radiation (123) from the radiation source (120) to produce at least one of the first radiation (122) or the second radiation (123).

5. The apparatus of claim 1, wherein the radiation subsystem (114) further comprises a detector (116) for detecting characteristics of at least one of the first radiation (122) or the second radiation (123).

6. The apparatus of claim 1, wherein the radiation source (120) is configured to produce the first radiation (122) and the second radiation (123) with an intensity distribution that varies in time.

7. The apparatus of claim 1, wherein the radiation source (120) is configured to produce the first radiation (122) and the second radiation (123) sequentially.

8. The apparatus of claim 1, wherein the radiation source (120) is configured to produce the first radiation (122) and the second radiation (123) simultaneously.

9. The apparatus of claim 1, wherein the radiation source (120) is configured to produce the first radiation (122) and the second radiation (123) in a temporally offset way, such that they partially overlap in time.

10. The apparatus of claim 1, wherein the radiation source (120) is configured to produce at least one of the first radiation (122) or the second radiation (123) periodically in time.

11. The apparatus of claim 1, wherein the array of pixels (408) is a two-dimensional array of pixels (408).

12. The apparatus of claim 1, wherein the sensing subsystem (126) further comprises a variable filter (128) for selectively prohibiting portions of at least one of the fluorescent radiation (124) or the reflected radiation (125) from achieving the imager (130).

13. The apparatus of claim 1, wherein the control subsystem (132) is configured to classify patterns (402, 404, 406) in at least one of the fluorescent or reflected radiation image (400), including a percentage of at least one of the fluorescent or reflected radiation image (400) individually represented by each one of the patterns (402, 404, 406).

14. The apparatus of claim 1, wherein the control subsystem (132) is configured to classify patterns (402, 404, 406) in at least one of the fluorescent or reflected radiation image (400), including an orientation of each one of the patterns (402, 404, 406), where the orientation is at least one of horizontal, vertical, and non-ordinal.

15. A method for optically characterizing a textile sample (106), the method comprising the steps of:
presenting the sample (106) against a viewing window (108) with a presentation subsystem (102),
directing a desired first radiation (122) in the ultraviolet range of the electromagnetic spectrum and a desired second radiation (122) in the visible range of the electromagnetic spectrum from a radiation source (120) through the viewing window (108) toward the sample (106), thereby causing the sample (106) to produce a fluorescent radiation (124) and a reflected radiation (125),
capturing the fluorescent radiation (124) and the reflected radiation (125) with a sensing subsystem (126) including an imager (130) in an array of pixels (408), where each pixel (408) records an intensity of both the fluorescent radiation (124) and the reflected radiation (125) at the pixel location, and
with a processor (136), controlling the presentation subsystem (102), the radiation subsystem (114), and the sensing subsystem (126), creating a fluorescent and reflected radiation image (400) containing both spectral information and spatial information in regard to the fluorescent radiation (124) and the reflected radiation (125) of the sample (106), and processing the fluorescent and reflected radiation image (400) into a brightness image
$$BI ( {λ}_{j} ) = a ( {λ}_{j} ) * FI [ \begin{matrix}{i}_{11} & … & {i}_{1 n} \\ ⋮ & {i}_{xy} & ⋮ \\ {i}_{m 1} & … & {i}_{mn}\end{matrix} ] + b ( {λ}_{j} ) * LI [ \begin{matrix}{i}_{11} & … & {i}_{1 n} \\ ⋮ & {i}_{xy} & ⋮ \\ {i}_{m 1} & … & {i}_{mn}\end{matrix} ] ,$$
where:
a(λⱼ) is weighting factor for the fluorescence image of wavelengths, where λₘᵢₙ<Δⱼ<λₘₐₓ, j=1, 2, . . . , k,
b(Δⱼ) is weighting factor for the reflected radiation image of wavelengths, where λₘᵢₙ<Δⱼ<λₘₐₓ, j=1, 2, . . . , k,
BI(λⱼ) is the brightness image of the sample 106 for one or more ranges of wavelengths, where λₘᵢₙ<Δⱼ<λₘₐₓ, j=1, 2, . . . , k,
FI(λⱼ) is the fluorescence image of the sample 106 for one or more ranges of wavelengths, where λₘᵢₙ<Δⱼ<λₘₐₓ, j=1, 2, . . . , k,
LI(Δⱼ) is the reflected radiation image of the sample 106 for one or more ranges of wavelengths, where λₘᵢₙ<Δⱼ<λₘₐₓ, j=1, 2, . . . , k,
n is the number of spatial points for the image in the horizontal direction,
m is the number of spatial points for the image in the vertical direction, and
i_{xy} is the energy level of the sample at coordinate location of x and y
at wavelength Δⱼ.

16. The method of claim 15, further comprising pressing the sample (106) against the viewing window (108) with a press (104).

17. The method of claim 15, further comprising at least one of shaping or filtering first radiation (122) and the second radiation (123) from the radiation source (120) with optics (118) before at least one of the first radiation (122) or the second radiation achieves the sample (106).

18. The method of claim 15, further comprising detecting characteristics of at least one of the first radiation (122) or the second radiation (123) with a detector (116).

19. The method of claim 15, further comprising producing the first radiation (122) and the second radiation (123) with an intensity distribution that varies in time.

20. The method of claim 15, further comprising producing the first radiation (122) and the second radiation (123) sequentially.

21. The method of claim 15, further comprising producing the first radiation (122) and the second radiation (123) simultaneously.

22. The method of claim 15, further comprising producing the first radiation (122) and the second radiation (123) in a temporally offset way, such that they partially overlap in time.

23. The method of claim 15, further comprising producing at least one of the first radiation (122) or the second radiation (123) periodically in time.

24. The method of claim 15, further comprising selectively prohibiting the fluorescent radiation (124) and the reflected radiation (125), respectively, from achieving the imager (130).

25. The method of claim 15, further comprising classifying patterns (402, 404, 406) in at least one of the fluorescent or reflected radiation image (400), including a percentage of at least one of the fluorescent or reflected radiation image (400) individually represented by each one of the patterns (402, 404, 406).

26. The method of claim 15, further comprising classifying patterns (402, 404, 406) in at least one of the fluorescent or reflected radiation image (400), including an orientation of each one of the patterns (402, 404, 406), where the orientation is at least one of horizontal, vertical, and non-ordinal.
